(19) United States
(12) Patent Application Publication
(10) Pub. No.: US 2025/0180264 A1
(43) Pub. Date: Jun. 5, 2025

(54) REFRIGERANT TESTING

(71) Applicant: Edwards Vacuum LLC, Sanborn, NY (US)

(72) Inventor: Kaustubh Kale, Chelmsford, MA (US)

(21) Appl. No.: 18/845,710

(22) PCT Filed: Mar. 1, 2023
(86) PCT No.: PCT/IB2023/051900

§ 371 Date: Sep. 10, 2024

(30) Foreign Application Priority Data
Mar. 11, 2022 (GB) 2203417.7

(51) Int. Cl. F25B 49/02

(52) U.S. Cl. CPC F25B 49/02; F25B 2700/19; F25B 2700/2108

(57) ABSTRACT

A refrigerant testing device and method for testing refrigerant diverted from a refrigeration system. The method comprises: diverting refrigerant from said refrigeration system to a container; changing a temperature of said refrigerant within said container; measuring a pressure and temperature of said refrigerant as said temperature changes; comparing said measured pressure and temperature values with reference values and where said comparing indicates a difference between said measured values and said stored reference values of more than a predetermined amount, generating a warning indication.

### CROSS-REFERENCE TO RELATED APPLICATION

This application is a Section 371 National Stage Application of International Application No. PCT/IB2023/051900 filed Mar. 1, 2023, and published as WO 2023/170519 A1 on Sep. 14, 2023, the content of which is hereby incorporated by reference in its entirety and which claims priority of British Application No. 2203417.7, filed Mar. 11, 2022.

### FIELD

The field of the invention relates to testing of refrigerant.

### BACKGROUND

Closed loop refrigeration systems generally contain a set amount of working fluid (also called refrigerant or gas here onwards). These systems can operate on any one or combination of thermodynamic cycles such as, but not limited to Joule Thomson Cycle, GM Cycle, Stirling Cycle etc. The refrigerant in these systems may be a single compound or a mixture of several different compounds.

Because the system is closed, that is there is no mass of working fluid entering or exiting the system, the performance of such systems is very sensitive to the amount of working fluid contained in them. It is popular practice to charge (fill) these systems with the exact amount of working fluid required to obtain optimum performance. If due to any reason (manufacturing defect, installation error, damage to equipment, operator error etc.) the system loses any part of the working fluid, that is the working fluid leaks out of the system, or composition of the working fluid changes, the performance of the system may be severely affected. This generally leads to a detrimental effect on system performance and if not observed in time, can lead to overall system failure and require time consuming and expensive measures to fix the system. Furthermore, several refrigeration systems contain working fluids that are harmful to the environment, and as such it is illegal to release these gases to atmosphere. If there is a leak in such a system, the owner/manufacturer/operator can also be liable to legal consequences, per applicable environmental protection legislation.

For the above reasons, it is important to be able to monitor the health of the refrigerant charge in a system (during operation) in order to detect any potential problems so that corrective actions can be taken in time. Problems may arise due to deterioration of the refrigerant charge, loss of refrigerant charge from the system, and/or a change in composition of mixed refrigerant charge.

The discussion above is merely provided for general background information and is not intended to be used as an aid in determining the scope of the claimed subject matter. The claimed subject matter is not limited to implementations that solve any or all disadvantages noted in the background.

### SUMMARY

A first aspect provides a refrigerant testing device for testing refrigerant diverted from a refrigeration system, said refrigerant testing device comprising: a container, said container comprising an inlet for receiving said refrigerant from said refrigeration system and an outlet for returning said refrigerant to said refrigeration system; an inlet flow control device for opening and closing said inlet; and an outlet flow control device for opening and closing said outlet; a temperature sensor for sensing a temperature of said refrigerant within said container; a pressure sensor for sensing a pressure of said refrigerant within said container; a temperature controller for controlling a temperature of said refrigerant within said container; control circuitry configured to control said refrigerant testing device to test said refrigerant by: controlling said inlet and outlet flow control devices to admit said refrigerant into said container and to isolate said refrigerant within said container; control said temperature controller to change a temperature of said refrigerant; control said temperature and pressure sensors to measure said temperature and said pressure of said refrigerant as said temperature changes; and analysing circuitry configured to compare values of said measured temperature and pressure with reference values; and to indicate an error where said measured pressure and temperature values differ from said reference values by more than a predetermined amount.

It was recognised that deterioration and/or leaks of the working fluid in a refrigeration system has a significant impact on performance, which performance will continue to degrade if the problem is not identified or addressed. It was therefore determined that it would be advantageous if the working fluid could be monitored in-situ or at least quasi in-situ so that changes to the properties/amount of working fluid can be identified.

This has been addressed with the use of a refrigerant testing device configured to divert a small amount of refrigerant from a refrigeration system to a testing chamber or container. The container has a predetermined volume and the testing of the fluid involves changing the temperature of the fluid across a temperature range while monitoring how the pressure and temperature vary. As the container has a predetermined set volume, the changes in pressure can be related directly to the changes in temperature. These changes can be compared with reference values to determine whether they are as expected or within predetermined limits of the reference values. Where they are not then a warning may be indicated. This warning may be a simple “fault detected” type warning or it may include information such as data collected during the testing, which data may provide some indication of the fault.

In this regard where the pressure is higher than expected then this may indicate deterioration or decomposition of (one of) the refrigerants. Where it is lower than expected then this may be indicative of a leak or it may again be indicative of deterioration of refrigerant. In this regard depending on the nature of the refrigerant and the temperature at which the unexpected pressure reading occurs, it may be possible to isolate the probable cause of the unexpected pressure reading from the multiple possible ones.

In some embodiments, said inlet is configured to connect to a portion of a refrigerant line within said refrigeration system and said outlet is configured to connect to a further portion of said refrigerant line within said refrigeration system, said further portion being at a lower pressure than said portion.

In some embodiments, said refrigerant testing device is configured to test a mixed refrigerant, said temperature controller being configured to control said temperature to change across a temperature range where at least one component of said mixed refrigerant changes between a liquid and a gaseous state.

Where the refrigerant is a mixed refrigerant then controlling the temperature controller to control the temperature to change across a temperature range which includes a phase change of at least one of the components of the mixed refrigerant allows further details regarding the different components of the mixed refrigerant to be determined. In this regard, differences in pressure compared to the reference pressure values either side of a phase change may indicate the contribution to this difference by a particular component and may allow a more complete diagnosis of the fault to be made. In this regard, certain refrigerants may be more prone to decomposition than others, while some may be more prone to escape if there is a small leak, thus determining which of the refrigerants may be causing the unexpected result may help diagnose what the problem may be.

As is discussed in more detail below, in some embodiments the refrigerant is sampled from more than one location and in such a case it may be advantageous to sample the mixed refrigerant at one location where all the components are gaseous and the mixed refrigerant is tested across a temperature range where they remain as gases and to sample the mixed refrigerant at another location in the refrigeration system where at least one of the components is a liquid on sampling and changes to a gaseous state during testing.

In some embodiments the refrigerant testing device comprises a data store said data store storing said reference values.

It should be noted that although the temperature controller may be a cooler and/or a heater in some embodiments the temperature controller comprises a heater this being an inexpensive and space efficient way of controlling temperature.

In some embodiments, said analysing circuitry is configured to analyse said changes in pressure at different temperatures and to determine information regarding a composition of said mixed refrigerant from said changes.

In some embodiments, said control circuitry is configured to control said refrigerant testing device to test said refrigerant periodically.

As noted previously, determining problems with the working fluid in a refrigeration system is important and thus, it may be advantageous where a refrigerant testing device is present to control that device to test the refrigerant periodically. This may be periodically in time or it may be periodically in use of the refrigeration system. For example, it may be tested after a particular process has been performed or at a particular point in a process of the refrigeration system.

In some embodiments, said refrigerant testing device comprises two or more containers each comprising an inlet and outlet and corresponding inlet and outlet flow control devices, said inlets and outlets being configured for connection to different locations within said refrigeration system, said control circuitry being configured to compare said changes in pressure and temperature with said reference values for said refrigerant in each of said containers; and to indicate an error where said changes in pressure and temperature differ from said reference values by more than a predetermined amount in one or more of said containers.

Although, the refrigerant testing device may include a single container, in some cases it may include two or more containers. These can be arranged such that the refrigerant that they sample is diverted from different portions of the refrigeration system. Changes in the refrigerant properties that are extracted from different portions of the refrigeration system may provide further diagnostic information on the performance of the refrigeration system and on where and/or which part or component of the mixed refrigerant is particularly affected by the problem.

In this regard, the inlets and outlets are attached to different portions of the refrigerant lines such that the refrigerant is sampled at different points in the refrigeration cycle.

In some embodiments, said analysing circuitry comprises a machine learning algorithm configured to analyse said measured pressure and temperature values and signals received from said refrigeration system indicative of its operation; to determine from said signals received and from previously measured pressure and temperature values expected pressure and temperature values; and to update said reference values with said expected pressure and temperature values.

Setting the initial reference values accurately can be difficult, particularly where the refrigerant testing device is not connected to the refrigeration system at manufacture. Even where the refrigerant testing device is part of the refrigeration system, the system to be cooled and thus, the load on the system is only present once the system is being utilised and may not be able to be tested at manufacture and this will affect the pressure and temperature of the sample taken at a particular point and during a particular operation. Providing a machine learning algorithm that can analyse the measured values over time in conjunction with signals received from the refrigeration system indicative of temperature, pressure at different places in the system and mode of operation of the system when the values were measured allows the machine learning algorithm to determine changes in pressure and temperature values that it deems not to be indicative of changes in the refrigerant and this allows the machine learning algorithm to update the reference values and in some cases the predetermined amounts.

In some embodiments, said machine learning algorithm is configured to analyse said measured pressure and temperature values during an initial period following commencement of said refrigerant testing and to derive said expected pressure and temperature values from said analysed values.

As noted previously one problem in setting the reference values is that the refrigerant testing device may not be attached to the refrigeration system or the refrigeration system may not be attached to the system that is being cooling when these values are set. The measured pressure and temperature values during the early part of operation of the refrigerant testing device may provide a good indication of the expected values prior to any leaks or refrigerant decomposition and thus, they may be used by the machine learning algorithm to update the reference values.

A further aspect provides a refrigeration system comprising: a refrigerant supply line for supplying refrigerant to an evaporator; a refrigerant return line for returning refrigerant from said evaporator; a compressor for compressing refrigerant received from said return line; and a refrigerant testing device according to a first aspect.

In some embodiments, said refrigerant testing device inlet is coupled to said refrigerant supply line and said outlet to said refrigerant return line.

In some embodiments, said control circuitry is configured to control said refrigerant testing device to perform an initial step of generating and storing said reference values for said refrigeration system by controlling said refrigerant device to test said refrigerant a predetermined number of times and to generate said reference values from said results.

Although, the reference values may be preloaded into the refrigerant testing device, generally they are generated by the device itself during an initial calibration type step whereby refrigerant within the refrigeration system is tested a number of times, generally when the system is new, and the values of temperature and pressure are measured and are used to generate the reference values. In this regard, the reference values may be generated from some type of average of these measured values, it may be a mean or it may be a mode value that is taken.

In some embodiments, the predetermined amount that triggers the error indication may be generated from this initial calibration measurements and in particular from measured differences in the values that are measured during the different initial tests. Where there is not much difference in these measured values, then it may be expected that even a small variation in the measured values will be indicative of some sort of change in the working fluid, whereas where there is a larger difference in the measured values during the calibration tests then clearly a higher predetermined amount will be needed as the threshold value.

In some embodiments, an initial step is performed for a plurality of different refrigeration system operating conditions or operating modes to generate a plurality of corresponding reference values.

In some cases, the reference values may be generated during a plurality of different operating conditions and/or modes and corresponding reference values generated accordingly. When the refrigerant is tested during operation then the testing will be performed during these different modes of operation and the relevant reference values used for the comparison. Again these additional tests may provide further diagnostic information when some unexpected values are measured.

In some embodiments, said control circuitry is configured to control said refrigerant testing device to perform said initial step in both of said containers such that said reference values are generated and stored for each of said two or more containers.

Where the refrigerant testing device has more than one container or test chamber, then reference values may be generated that are applicable to each container by performing the initial calibration measurements for each of the containers. During the refrigerant testing, the relevant reference values will be used in the comparisons depending on which container the measured values are derived from.

In some embodiments, the comparison step may not be a comparison of actual values but more a comparison of trends or curves.

Although the first container and second container may be coupled to any two distinct locations within the refrigeration system, in some embodiments, said refrigeration system further comprises at least one phase separator and at least one heat exchanger; a first inlet to a first container being coupled to said refrigerant supply line at a first point and a second inlet to a second container being coupled to said refrigerant supply line at a second point, said first and second being separated by at least one of a phase separator and a heat exchanger.

In some embodiments, a working fluid of said refrigeration system comprises a mixed refrigerant.

In some embodiments, the sampling points may be selected to be points where the temperature and pressure and where the refrigerant is a mixed refrigerant the composition are stable.

Where there are two sampling points and a mixed refrigerant then the first and second points may be selected to be at locations where the temperature, pressure and/or composition of the mixed refrigerant are different. These two points may be at different portions of the refrigerant line at different points in the refrigeration system. In some embodiments, one may be taken before and one after a phase separator such that certain components of the mixed refrigerant may be more plentiful in one of the samples than in the other, this allows differences between the reference values and the measured samples to be informative not only of a fault but of which particular component of the refrigerant may not be as expected.

In some embodiments, said control circuitry is configured to determine operation of said refrigeration system and to control said refrigerant testing device to monitor said refrigerant during a particular mode of operation of said refrigeration system and in some cases during a plurality of different modes of operation of said refrigeration system.

A further aspect provides a method of testing refrigerant within a refrigeration system, said method comprising: diverting refrigerant from said refrigeration system to a container; changing a temperature of said refrigerant within said container; measuring a pressure and temperature of said refrigerant as said temperature changes; comparing said measured pressure and temperature values with reference values and where said comparing indicates a difference between said measured values and said reference values of more than a predetermined amount, generating a warning indication.

In some embodiments, the method comprises a first step of fitting a refrigerant testing device according to a first aspect to a refrigeration system.

Although the refrigeration system may be manufactured with the refrigerant testing device attached to it, in some embodiments the refrigerant testing device may be retro-fitted to an existing refrigeration system to improve that system and allow the quality/amount of refrigerant to be continually monitored.

In some embodiments, the method comprises prior to testing said refrigerant generating said reference values by: performing said method for testing said refrigerant a predetermined number of times and generating said reference values from said measured pressure and temperature values.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

The summary is provided to introduce a selection of concepts in a simplified form that are further described in the detailed description. This summary is not intended to identify key features or essential features of the claimed subject matter, nor is it intended to be used as an aid in determining the scope of the claimed subject matter.

### BRIEF DESCRIPTION OF THE DRAWINGS

Embodiments of the present invention will now be described further, with reference to the accompanying drawings, in which:

FIG. 1 shows a refrigerant testing device according to an embodiment;

FIG. 2 shows a refrigeration system according to an embodiment;

FIG. 3 shows a flow diagram illustrating steps in a method according to an embodiment;

FIG. 4 shows a flow diagram illustrating steps in a method of calibration of a refrigerant testing device; and

FIG. 5 shows steps in a method for updating reference values.

### DETAILED DESCRIPTION

Before discussing the embodiments in any more detail, first an overview will be provided.

Embodiments provide a method and appratus to regularly monitor the health of the refriergant charge in a system. Embodiments work on the principle that a known gas or mixture of gases exhibit consistent temperature and pressure charatersites based on the thermodynamic properties of the gas/gases. Applying the thermodymanic relation between pressure and temperature for a given volume of gas, the quantitiy (and composition) of the gases in the volume can be determined. By employing this technique at suitable locations in the system, the overall health of the refrigerant charge of the system can be extrapolated from the measurements.

Embodiments works on the principle that for a known gas or mixture of gases, the P, V and T (pressure volume and temperature) properties of the gas are inter-related and can be predicted using thermodynamic equations of state.

Embodiments utilize the principal of periodically diverting a small portion (generally less than 5%) of the refrigerant charge in the system to a specific test part of the system (called test volume). The test volume may be an integral part of the system, such that it does not need to be connected/disconnected for taking measurements. In this regard it may be a part of the system when the system is manufactured, or it may be added to the system as an additional component post manufacture. This test volume can be a tank, pressure vessel, container or similar and its geometry and internal volume is selected depending on the size and type of the overall system.

This specific test part (test volume) of the system is maintained at a set temperature and pressure and also the inlet and outlet of this volume are maintained at predetermined P and T values. Flow control devices (manual or powered) such as valves are used to control the flow of gas into and out of this volume. By controlling the time (in open position), inlet geometry and inlet pressure of the flow control devices, a predetermined quantity of the working fluid can be diverted to this test volume. Once the working fluid is isolated in this volume, the temperature of the working fluid can be accurately altered/controlled using heaters or cooling, as required.

The test volume in consideration has temperature and pressure monitoring apparatus (thermocouples, diodes, pressure transducers etc.) that can measure accurately the temperature and pressure of the working fluid within the container. For a given working fluid (or mixture) as the temperature changes, the pressure will also change in a manner that is predictable and repeatable.

The temperature and pressure values for a particular working fluid (or mixture) in this test volume can be tabulated for a variety of system operating conditions, operating modes, states etc. to generate electronic tables or plots of the T and P relationship. This is best accomplished when the system is new and is free of wear, defects, leaks etc. that can occur after continued operation. Once T and P reference values and/or relationships are established for all system performance modes, these can be used as baseline reference values or curves for future comparison.

An example of implementation for a refrigeration system in use, a part of the working fluid is diverted to the measurement volume using the flow control equipment described above. The amount of working fluid diverted is small enough so it does not affect the performance of the system. This working fluid now captured in the test volume is now either heated or cooled across a temperature range to a predetermined temperature. The pressure of the working fluid is measured while its temperature is raised and/or lowered and also when it has reached a final temperature that it is heated or cooled to, the set-point temperature. For a given operating state of the system, the pressure and temperature values of the working fluid in the test volume should follow very closely the baseline reference values established and stored when the system was manufactured/installed/initialised.

In some embodiments the above method is performed periodically, and the trend of the P and T values be observed. If the trend of P and T values falls outside predetermined acceptable limits, it can be concluded that there is a problem with the working fluid. This can signal a leak in the system, change in charge composition, degradation/deterioration of one or multiple constituents of the charge etc. Every scenario however negatively affects system performance and thus is important to detect. At this point, the system's controller can provide an alarm/error message to draw attention to this issue. When caught in time, expensive repairs and down time of the refrigeration system can be avoided or at least reduced.

In some embodiments, such as where a mixed refrigerant is used, it is advantageous to divert working fluid to the test volume from a location in the system where there is a low variation in the inlet T, P and composition of the working fluid between multiple samples. In a system that operates on a single refrigerant/working fluid, this is an easier task as one must control only for T and P of the fluid. In a system that operates on a mixture of several gases however, it is important to divert the working fluid from such a location/s in the system where the composition of the fluid is most consistent for any given duty cycle of the system. A system designer can identify such locations in their design to ensure consistency of measurement.

Another aspect of this invention is how the baseline T and P values are obtained/determined for a given system. The following approaches or any combination of the following are possible:
1. The baseline T and P values can be recorded by running the system in all possible operating modes and diverting working fluid to the test volume in each operating mode several times to establish baseline and acceptable errors bands can be established.
2. T and P values can be predicted using thermodynamic equations. In this approach the working fluid is diverted in the test volume at one particular operating mode of the system, and this is repeated several times to establish a baseline for that operating mode. Once this is established, T and P baseline values for different modes can be calculated using thermodynamic equations.
3. ML machine learning and Al artificial intelligent techniques can be used in conjunction with curves/data and thermodynamic equations to create and train intelligent ‘models’ to predict the T and P for the sample working fluid for comparison.

In summary embodiments seek to extrapolate the health of the overall refrigerant charge of the system based on a small sample of working fluid taken in some embodiments periodically during operation of the system. This is especially advantageous for systems with mixed-refrigerants as the working fluid.

In a mixed refrigerant system, the composition of the working fluid changes depending on the physical location of the fluid mixture in the system, as well as the duty cycle of the cycle (idle, no load, full load etc.). It is proposed that the refrigerant mixture is sampled at specific locations in the system where it is known that the composition will be consistent. This sample is then isolated in an environment where the temperature of the sample is altered to such a value that variations due to phase change and similar physical phenomenon (freezing point depression etc.) are eliminated, and the sample is essentially ‘homogenized/baselined’ for measurement. For example, the temperature may be set such that each component of a mixed refrigerant is in a gaseous state. Differences in the pressure between the sample when it is multi-phase and single phase are then compared with the corresponding reference values, to provide additional information.

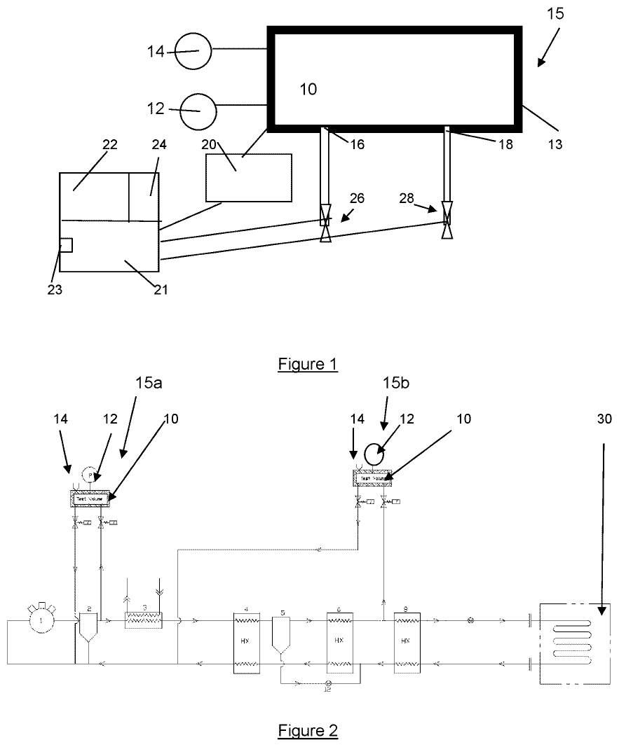

FIG. 1 shows a refrigerant testing device 15 according to an embodiment. Refrigerant testing device 15 comprises a container 10 of a predetermined volume that is insulated to reduce variations in the temperature and which in this embodiment is surrounded by a heating element 13 that is controlled by temperature control device 20. Container 10 comprises an inlet 16 for admitting refrigerant from a refrigeration system and an outlet 18 for returning refrigerant to the refrigeration system. Inlet 16 has an inlet channel connected to it with a valve 26 for controlling the flow of the refrigerant while outlet 18 has an outlet channel with a valve 28. Valves 26 and 28 and temperature control circuitry 20 are controlled by control circuitry 21. Control circuitry 21 in some embodiments comprises an input port 23 for receiving signals from the refrigeration system. These signals may indicate the current mode of operation of the refrigeration system and/or may comprise measurements from temperature, pressure and flow sensors within the refrigeration system.

Control circuitry 21 controls the refrigerant testing device to sample refrigerant and then test it by controlling valves 26 and 28 to allow refrigerant into the testing chamber or container 10 and then to seal it within the container 10. Temperature controller 20 controls the temperature of the refrigerant within the container 10 by controlling heater 13 to heat the container such that it warms up from an initial low temperature to a predetermined set higher temperature. Temperature sensor 14 and pressure sensor 12 monitor the temperature and pressure of the refrigerant as it is warmed up. Values from these sensors are transmitted to analysing circuitry 22 that compares the measured values with reference values stored within data store 24 and determines whether the values are as expected or whether the values indicate an anomaly.

Where they are not as expected then a warning indication may be generated. This warning indication may simply be a binary indication such as the illumination of a light or generation of a sound, or it may also comprise data from the measurements which may provide an indication of the type of anomaly that has been detected. For example, whether the pressure is higher than expected or lower than expected and the temperatures at which the unexpected pressure reading occurred are all indicative of the type of fault. A higher pressure may indicate a refrigerant that is decomposing, while a lower pressure may indicate a leak in the system.

In some embodiments the refrigerant is a mixed refrigerant with different components. In such a case the combination of temperature and pressure readings may provide an indication of the component(s) in the refrigerant that is responsible for the unexpected result. In this regard, where the pressure is measured across a temperature range where at least one of the mixed refrigerant components changes state then the contribution of this component to any anomalous result may be derived. In this regard one or more components of the refrigerant might be more prone to deterioration or decomposition such that the pressure may increase when these components are in the gaseous state, while one or more components may be more prone to leaking leading to a lower pressure than expected.

In some embodiments, where signals are received from the refrigeration system at input 23, then control circuitry 21 may control the refrigerant testing device 15 to perform the testing routine on the refrigerant at particular times during particular modes of operation of the refrigeration system. Where this is the case, data store 24 may store reference values of pressure and temperature that are relevant for these different modes of operation and the comparisons of the measured values will be performed against the relevant reference values. In some embodiments the refrigerant testing device may sample and test the refrigerant a number of times and average the results before doing the comparison and determining whether the refrigerant is performing as expected or not.

The reference values stored in data store 24 may be generated during an initial calibration step where the control circuitry controls the refrigerant testing device to sample the refrigerant, to change the temperature and to measure variations in pressure and temperature. It may do this a predetermined number of times and then generate reference values from the average of these measured values and generate threshold values which indicate by how much a measured value may vary from a reference value before an anomaly is indicated. The thresholds may be generated in dependence on the variation of the values measured during the calibration step. The calibration step may be performed during different modes of operation of the refrigeration system to generate reference values for each mode, and it may be performed in different testing chambers where there are plural testing chambers, reference values for each testing chambers being generated.

In some embodiments analysing circuitry 22 comprises a machine learning algorithm. The machine learning algorithm may receive signals from the refrigeration system in addition to the measured values of temperature and pressure. The machine learning algorithm may analyse the measured values of pressure and temperature in conjunction with the signals from the refrigeration system and identify differences and trends in the measured values. This machine learning algorithm may be used to update the reference values stored in data store 24 where it determines that the values measured differ consistently from the reference values and in a way that seems to be not due to changes in refrigerant properties.

Although in this embodiment, the refrigerant testing device has a single testing chamber or container 10 in some embodiments there may be a plurality of these with the inlet and outlet channels being connected to different parts of the refrigeration system to sample refrigerant at different places in the system. In these embodiments reference values relevant for each location are used.

FIG. 2 shows a refrigeration system according to an embodiment. This refrigeration system is a mixed refrigerant Joule-Thompson cryochiller. This cryochiller comprises a compressor 1 that compresses refrigerant received from the return refrigerant line and sends it along the refrigerant supply line towards evaporator 30. There are separators 2 and 5 that separate oil or liquid from the higher pressure supply line and send it to the lower pressure return line and there are heat exchanges 3, 4, 6 and 9 that act to cool the compressed refrigerant prior to it reaching the evaporator 30. In evaporator 30 the liquid refrigerant will expand to form a gas and will generate the cooling effect.

In this embodiment there are two refrigerant testing devices 15a, 15b each with a container 10 of a predetermined volume that is insulated and temperature controlled and each with an associated pressure sensor 12 and temperature sensor 14. There are valves on the inlets and outlets of the containers for controlling flow into and out of the containers 10. Control circuitry (not shown) controls these valves such that refrigerant is diverted from the refrigerant supply line into the test container and following testing sent back to the refrigerant return line. On initial sampling, both inlet and outlet valves may be left open to flush the chamber and then the outlet valve may be closed while the inlet valve remains open allowing the refrigerant to be sampled. Both valves may then be closed to isolate the sample from the refrigeration system. Temperature control circuitry associated with the container 10 then changes the temperature of the container 10 across a range of temperatures that in this embodiment are sufficient for at least some components of the mixed refrigerant in at least one of the containers to change state. The temperature and pressure of the refrigerant during these temperature changes are measured and compared with the stored reference values to determine the condition of the refrigerant in the refrigeration system.

In this embodiment, the refrigerant is sampled at two locations, location 1 and location 2. Location 1 being close to the compressor and having the refrigerant at a higher temperature while location 2 is further from the compressor. The refrigerant at location 2 is cooler than the refrigerant at location 1 and thus, may have a different phase composition of the mixed refrigerant on sampling. Differences in the measurements and an analysis of the measurements from the two locations may provide additional information when diagnosing any fault.

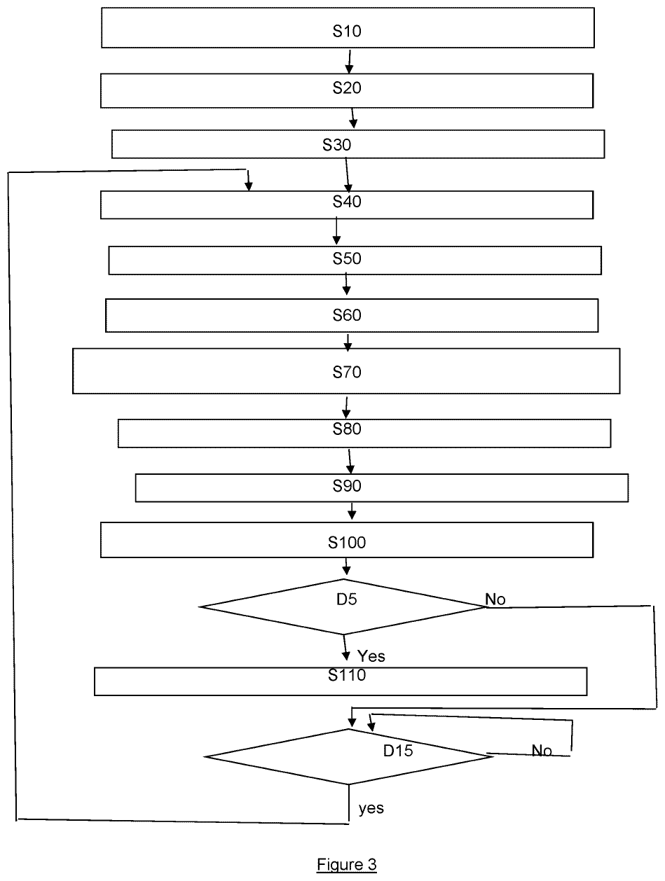

FIG. 3 schematically shows steps in a method according to an embodiment. In this embodiment at step S10 the refrigerant testing device is attached to a refrigeration system. In some embodiments the refrigeration system may be manufactured with an integral refrigerant testing device, while in other embodiments the refrigerant testing device may be manufactured separately and retro fitted to an existing refrigeration system. In the latter case, step S10 is performed while in the former case this step is not required.

Step S20 is then performed where the refrigeration system is attached to a load. In this regard, the load may be a cooling circuit for a semiconductor wafer and the attachment of the refrigeration system to this load will affect the volume of and thus, the pressure in the refrigeration system.

At step S30 refrigeration is initiated in the refrigeration system and at step S40 the refrigerant is sampled by controlling the inlet and outlet valves to the sample chamber to admit refrigerant into the sample chamber. It is isolated within the sample chamber at step S50.

The refrigerant testing regime is then started on the isolated sample by, at step S60, the temperature of the refrigerant being changed across a temperature range. This may be done by heating or cooling the refrigerant across the temperature range. Where the refrigerant is a mixed refrigerant then this temperature range may be selected so that at least one of the components of the refrigerant changes state between a gas and a vapour during the temperature change. In some embodiments, the change in temperature may lead to a homogenized mixed refrigerant, i.e., all components are in the same state.

The temperature and pressure of the isolated refrigerant is measured as the temperature changes at step S70. In some embodiments, the testing steps S40 to S70 are repeated one or more times at step S80 and at step S90 the results are averaged. In other embodiments, the results of a single test may be used without repeating the steps and averaging the results.

The results are then compared with stored reference values at step S100 and it is determined whether or not they differ from the stored reference values by more than a predetermined amount (thresholds) at step D5. If it is determined that they do then a warning is output at step S110 whereas if they do not then at step D15 it is determined whether a predetermined time has expired and when it has the testing steps are repeated.

In this way, refrigerant within a refrigeration system is periodically measured and where there are significant changes in the results compared with reference values a warning can be output and an early indication of refrigerant deterioration or leakage can be found prior to the system failing.

In some cases the refrigerant may be sampled at more than one location and the method steps S40 to S110 performed for each location. In some cases the refrigerant may be tested during different modes of operation of the refrigeration system and in this case different reference values and different threshold values relevant to the different modes may be used.

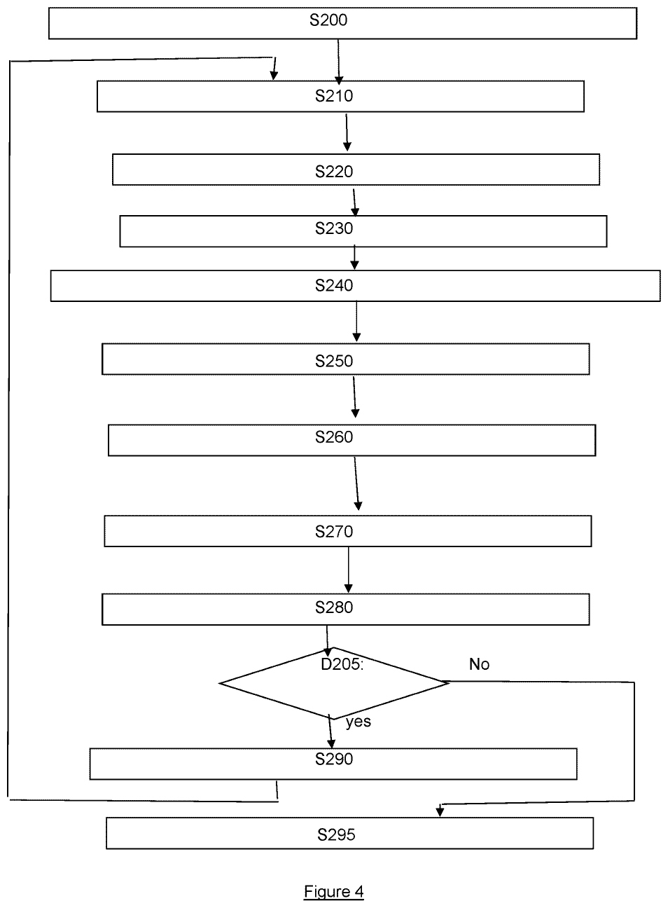

FIG. 4 shows a flow diagram illustrating steps performed when generating the reference values during an initial calibration procedure for a reference testing device according to an embodiment. In an initial step S200 a refrigeration system is turned on and controlled to perform one mode of refrigeration. In this regard the refrigeration system may operate in several different modes, such as cooling mode and standby mode. At step S210 refrigerant is sampled and at step S220 it is isolated. At step S230 the temperature of the isolated refrigerant is changed across a temperature range and at step S240 the temperature and pressure of the isolated refrigerant is measured as the temperature changes. At step S250 steps S210 to step S240 are repeated. Step S250 may itself be repeated a number of times. At step S260 the results from the repeated measurements are averaged and at step S270 reference values are generated from these averaged results. Threshold values are then generated at step S280. In this regard, the threshold values may depend on the variation in the measured values that were averaged to form the reference values. These reference and threshold values are then stored for use in the testing steps of the method of FIG. 3.

It is then determined at step D205 if all of the operational modes have been tested and if not then step S290 of control the refrigeration system to perform a further mode of operation is performed and steps S210 to S280 and D205 are again performed. If it is determined at step D205 that all operational modes have been tested then at step S295 the calibration procedure is ended.

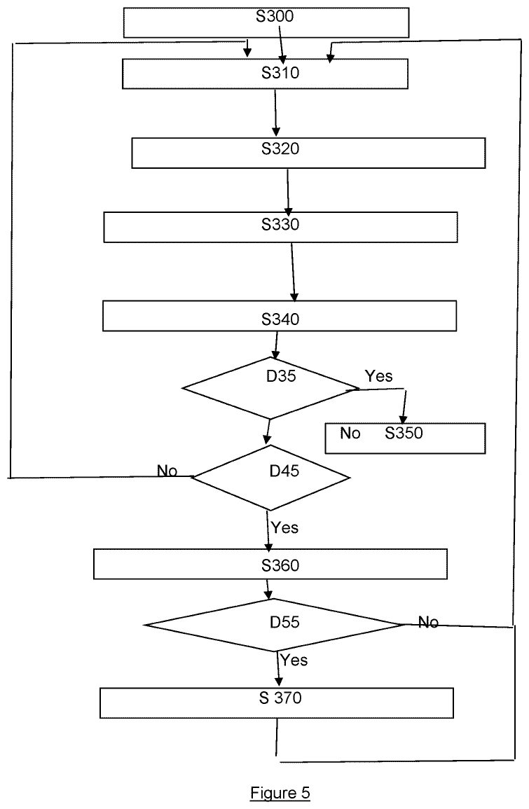

FIG. 5 schematically shows a method where the reference values may be updated to more accurately reflect the refrigerant within the refrigeration system. In some cases this may be done using a machine learning algorithm within the analysing circuitry of an embodiment. In an initial step S300 the load is attached to the refrigeration system and at step S310 the refrigerant is tested by performing the steps of sampling isolating and changing the temperature of the refrigerant while measuring the temperature and pressure. At step S320 the refrigerant tests are repeated and at step S330 the results are averaged. In this regard, depending on the embodiment the refrigerant tests may be repeated two or more times. At step S340 the average results are compared with stored reference values and it is determined at step D35 whether or not thresholds are exceeded, that is whether the measured values are different from the reference values by more than a predetermined amount. If they are then a warning is output at step 350 while if they are not the tests are repeated a certain number N times, thus step D45 determines whether or not the test has been repeated N times and if not it is repeated again and if so then the results are analysed . . .

The analysing circuitry analyses the results at step S360 and determine at step D55 whether there is a consistent difference between the results and the reference values albeit not one where thresholds are exceeded. It may also receive and consider inputs from the refrigeration system such as temperature and pressure readings taken at different points at the time that the refrigerant was sampled and it may include these in its determination as to whether or not the variation in results indicates a consistent difference of the measured values to the reference values at step D35. If it determines a consistent difference then it may update the reference values at step S370 by this consistent difference and continue to repeat the tests.

In some cases, this method may be performed during the early stages of use of the refrigeration system with a particular load as the reference values will have been generated without the load in place and thus, there may be some offset in the reference values that can be detected and corrected for by measurements taken in use. As the refrigeration system ages and it is more likely that the refrigerant will have leaked or decomposed then the machine learning algorithm may be configured to no longer update the reference values or to be more reluctant to do so, that is to require more data indicative of a change not being due to changes in refrigerant properties before it updates the values.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are described as example forms of implementing the claims.

1. A refrigerant testing device for testing refrigerant diverted from a refrigeration system, said refrigerant testing device comprising:
a container, said container comprising an inlet for receiving said refrigerant from said refrigeration system and an outlet for returning said refrigerant to said refrigeration system;
an inlet flow control device for opening and closing said inlet; and
an outlet flow control device for opening and closing said outlet;
a temperature sensor for sensing a temperature of said refrigerant within said container;
a pressure sensor for sensing a pressure of said refrigerant within said container;
a temperature controller for controlling a temperature of said refrigerant within said container;
control circuitry configured to control said refrigerant testing device to test said refrigerant by:
controlling said inlet and outlet flow control devices to admit said refrigerant into said container and to isolate said refrigerant within said container;
controling said temperature controller to change a temperature of said refrigerant;
controling said temperature and pressure sensors to measure said temperature and said pressure of said refrigerant as said temperature changes; and
analysing circuitry configured to compare values of said measured temperature and pressure with reference values; and to indicate an error where said measured pressure and temperature values differ from said reference values by more than a predetermined amount.

2. The refrigerant testing device according to claim 1, wherein said refrigerant testing device is configured to test a mixed refrigerant, said temperature controller being configured to control said temperature to change across a temperature range where at least one component of said mixed refrigerant changes between a liquid and a gaseous state.

3. The refrigerant testing device according to claim 2, wherein said analysing circuitry is configured to analyse said changes in pressure at different temperatures and to determine information regarding a composition of said mixed refrigerant from said changes.

4. The refrigerant testing device according to claim 1, wherein said control circuitry is configured to control said refrigerant testing device to test said refrigerant periodically.

5. The refrigerant testing device according to claim 1, wherein
said analysing circuitry comprises a machine learning algorithm configured to analyse said measured pressure and temperature values and signals received from said refrigeration system indicative of its operation;
to determine from said signals received and from previously measured pressure and temperature values expected pressure and temperature values; and
to update said reference values with said expected pressure and temperature values.

6. The refrigerant testing device according to claim 5, wherein said machine learning algorithm is configured to analyse said measured pressure and temperature values during an initial period following commencement of said refrigerant testing and to derive said expected pressure and temperature values from said analysed values.

7. The refrigerant testing device according to claim 1, wherein said refrigerant testing device comprises two or more containers each comprising an inlet and outlet and corresponding inlet and outlet flow control devices, said inlets and outlets being configured for connection to different locations within said refrigeration system, said control circuitry being configure to compare said changes in pressure and temperature with said reference values for said refrigerant in each of said containers; and
to indicate an error where said changes in pressure and temperature differ from said reference values by more than a predetermined amount in one or more of said containers.

8. A refrigeration system comprising:
a refrigerant supply line for supplying refrigerant to an evaporator;
a refrigerant return line for returning refrigerant from said evaporator;
a compressor for compressing refrigerant received from said return line; and
a refrigerant testing device according to claim 1.

9. The refrigeration system according to claim 8, wherein said refrigerant testing device inlet is coupled to said refrigerant supply line and said outlet to said refrigerant return line.

10. The refrigeration system according to claim 8, wherein said control circuitry is configured to control said refrigerant testing device to perform an initial step of generating and storing said reference values for said refrigeration system by controlling said refrigerant testing device to test said refrigerant a predetermined number of times and to generate said reference values from said results.

11. The refrigeration system according to claim 10, wherein initial step is performed for a plurality of different refrigeration system operating conditions or operating modes to generate a plurality of corresponding reference values.

12. The refrigeration system according to claim 10 where said refrigerant testing device is a refrigerant testing device comprising two or more containers each comprising an inlet and outlet and corresponding inlet and outlet flow control devices, said inlets and outlets being configured for connection to different locations within said refrigeration system, said control circuitry being configure to compare said changes in pressure and temperature with said reference values for said refrigerant in each of said containers and to indicate an error where said changes in pressure and temperature differ from said reference values by more than a predetermined amount in one or more of said container, wherein said control circuitry is configured to control said refrigerant testing device to perform said initial step in both of said containers such that said reference values are generated and stored for each of said two or more containers.

13. The refrigeration system according to claim 12, wherein said refrigeration system further comprises at least one phase separator and at least one heat exchanger;
a first inlet to a first container being coupled to said refrigerant supply line at a first point and a second inlet to a second container being coupled to said refrigerant supply line at a second point, said first and second points being separated by at least one of a phase separator and a heat exchanger.

14. The refrigeration system according to claim 8, wherein said refrigerant of said refrigeration system comprises a mixed refrigerant.

15. A method of testing refrigerant within a refrigeration system, said method comprising:
diverting refrigerant from said refrigeration system to a container;
changing a temperature of said refrigerant within said container;
measuring a pressure and temperature of said refrigerant as said temperature changes;
comparing said measured pressure and temperature values with reference values and where said comparing indicates a difference between said measured values and said stored reference values of more than a predetermined amount, generating a warning indication.

16. The method according to claim 15, comprising a first step of fitting a refrigerant testing device to a refrigeration system, said refrigerant testing device comprising:
a container, said container comprising an inlet for receiving said refrigerant from said refrigeration system and an outlet for returning said refrigerant to said refrigeration system;
an inlet flow control device for opening and closing said inlet; and
an outlet flow control device for opening and closing said outlet;
a temperature sensor for sensing a temperature of said refrigerant within said container;
a pressure sensor for sensing a pressure of said refrigerant within said container;
a temperature controller for controlling a temperature of said refrigerant within said container;
control circuitry configured to control said refrigerant testing device to test said refrigerant by:
controlling said inlet and outlet flow control devices to admit said refrigerant into said container and to isolate said refrigerant within said container;
controling said temperature controller to change a temperature of said refrigerant;
controling said temperature and pressure sensors to measure said temperature and said pressure of said refrigerant as said temperature changes; and
analysing circuitry configured to compare values of said measured temperature and pressure with reference values; and to indicate an error where said measured pressure and temperature values differ from said reference values by more than a predetermined amount..

17. The method according to claim 16 comprising prior to testing said refrigerant, generating said reference values by:
performing said method for testing said refrigerant a predetermined number of times and generating said reference values from said measured pressure and temperature values.
